(19) United States
(12) Patent Application Publication
(10) Pub. No.: US 2011/0263580 A1
(43) Pub. Date: Oct. 27, 2011

(54) METHOD FOR TREATING ALZHEIMER'S DISEASE AND RELATED CONDITIONS

(76) Inventor: Thomas Miller, Rahway, NJ (US)

(21) Appl. No.: 13/133,080

(22) PCT Filed: Dec. 4, 2009
(86) PCT No.: PCT/US09/66750

§ 371 Date: Jun. 24, 2011

Related U.S. Application Data
(60) Provisional application No. 61/121,718, filed on Dec. 11, 2008.

(51) Int. Cl. A61K 31/18; A61K 31/196; A61K 31/5415; A61P 25/28; A61K 31/351; A61K 31/433

(52) U.S. Cl. 514/226.5; 514/605; 514/459; 514/362; 514/562

(57) ABSTRACT

This invention relates to a method for treating or preventing diseases associated with the deposition of β-amyloid peptide in the brain, such as Alzheimer's disease, said method involving administration of a gamma-secretase inhibitor in an intermittent dosing regime to a patient in need thereof.

This invention relates to a method for treating or preventing diseases associated with the deposition of β-amyloid peptide in the brain, such as Alzheimer's disease, or of preventing or delaying the onset of dementia associated with such diseases.

Alzheimer's disease (AD) is the most prevalent form of dementia. Its diagnosis is described in the Diagnostic and Statistical Manual of Mental Disorders, 4^{th} ed., published by the American Psychiatric Association (DSM-IV). It is a neurodegenerative disorder, clinically characterized by progressive loss of memory and general cognitive function, and pathologically characterized by the deposition of extracellular proteinaceous plaques in the cortical and associative brain regions of sufferers. These plaques mainly comprise fibrillar aggregates of β-amyloid peptide (Aβ). Aβ is formed from amyloid precursor protein (APP) via separate intracellular proteolytic events involving the enzymes β-secretase and γ-secretase (see Selkoe, Physiol. Rev., 81(2), 741-766 (2001). Variability in the site of the proteolysis mediated by γ-secretase results in Aβ of varying chain length, e.g. Aβ(1-38), Aβ(1-40) and Aβ(1-42). N-terminal truncations such as Aβ(4-42) are also found in the brain, possibly as a result of variability in the site of proteolysis mediated by β-secretase. After secretion into the extracellular medium, Aβ forms initially-soluble aggregates which are widely believed to be the key neurotoxic agents in AD (see Gong et al, PNAS, 100 (2003), 10417-22), and which ultimately result in the insoluble deposits and dense neuritic plaques which are the pathological characteristics of AD.

Other dementing conditions associated with deposition of Aβ in the brain include cerebral amyloid angiopathy, hereditary cerebral haemorrhage with amyloidosis, Dutch-type (HCHWA-D), multi-infarct dementia, dementia pugilistica and Down syndrome.

The role of secretases, including that of γ-secretase, in the processing of amyloid precursor protein (APP) to form Aβ is well documented in the literature, and so inhibiting the processing of APP by γ-secretase is recognised as a likely means of treating or preventing AD and related conditions (a recent review of activity in this area is provided by Garofalo, Expert Opin. Ther. Patents (2008) 18(7), 693-703). However, attempts to develop such a treatment have been hampered by the fact that γ-secretase is active towards a number of different transmembrane proteins in addition to APP (for a review, see Pollack and Lewis, Current Opinion in Investigational Drugs (2005), 6(1), 35-47), with the result that inhibition of γ-secretase can lead to unwanted side effects as well as the desired interruption of APP processing. In particular, γ-secretase plays a crucial role in the Notch cell-signalling process, which itself plays a crucial role in cell-fate determination. The Notch receptor protein is a transmembrane protein which, in response to activation by the relevant ligand, undergoes intramembranous cleavage by γ-secretase, releasing the Notch intracellular domain (NICD) which can then migrate to the cell nucleus and modulate gene transcriptions (Pollack and Lewis, supra). An example of cell-fate determination influenced by Notch is the differentiation of proliferative epithelial cells in the gastro-intestinal (GI) tract, with the result that suppression of Notch processing leads to intestinal goblet cell metaplasia and severe GI toxicity (Searfoss et al, J. Biol. Chem. (2003), 278(46), 46107-46116; Wong et al, J. Biol. Chem. (2004), 279(13), 12876-12882; Milano et al, Toxicological Sciences, (2004), 82, 341-358; and Van Es et al, Nature (2005), 435, 959-963).

There is therefore a need for a means of inhibiting the processing of APP that has minimal effect on the processing of Notch. Although there are a few reports of γ-secretase inhibitors showing selectivity for APP processing over Notch processing (see Garofalo, supra; and Petit et al, J. Neuroscience (2003), 74, 370-377), none has so far led to a viable treatment for AD. Furthermore, the present investigators have found that certain compounds which show selectivity for APP processing over Notch processing in vitro can still cause GI toxicity in vivo, particularly when administered to higher species such as primates. There is therefore an ongoing need for improved methods for inhibiting APP processing without incurring significant Notch-related GI toxicity.

In a first aspect, the invention provides a method for treating or preventing a disease involving deposition of β-amyloid (Aβ) in the brain which comprises administering to a patient in need thereof a therapeutically effective amount of a gamma-secretase inhibitor (GSI) by an intermittent dosing regimen.

In a second aspect, the invention provides a pharmaceutical composition comprising a pharmaceutically acceptable carrier and a GSI for use in treating or preventing a disease involving deposition of β-amyloid (Aβ) in the brain by an intermittent dosing regimen.

In a third aspect, the invention provides the use of a GSI for the manufacture of a medicament for treating or preventing a disease involving deposition of β-amyloid (Aβ) in the brain by an intermittent dosing regimen.

The disease involving deposition of Aβ in the brain is typically Alzheimer's disease (AD), cerebral amyloid angiopathy, hereditary cerebral haemorrhage with amyloidosis, Dutch-type (HCHWA-D), multi-infarct dementia, dementia pugilistica or Down syndrome, in particular AD.

The term “intermittent dosing regimen” refers to repeating cycles of drug administration in which the drug in question is administered on one or more consecutive days (“days on”) followed by one or more consecutive days of rest on which the drug is not administered (“days off”). The cycles may be regular, in that the pattern of days on and days off is the same in each cycle, or may be irregular, but regular cycles in which the drug is administered on the same weekday(s) are more convenient for the patient.

In one embodiment the intermittent dosing regimen comprises a repeating cycle of GSI administration on 1 to 3 consecutive days followed by at least 4 days of rest.

In a sub-embodiment the intermittent dosing regimen comprises a repeating cycle of GSI administration on 3 consecutive days followed by 4 days of rest.

In an alternative sub-embodiment the intermittent dosing regimen comprises a repeating cycle of GSI administration on 1 day followed by 6 days of rest.

It is surprisingly found that a GSI, when administered to mammals via an intermittent regimen as defined above, can provide and maintain a therapeutically-useful level of inhibition of APP processing without incurring significant GI toxicity. This is true even when administration of the same GSI on a continuous daily dosing regimen, at a level sufficient to provide and maintain a therapeutically-useful level of inhibition of APP processing, causes serious GI toxicity, and even though the individual doses suitable for use on an intermittent dosing regimen are typically much larger (e.g. >50-fold) than the individual doses required by a continuous daily dosing regimen.

The term “γ-secretase inhibitor” (GSI) refers to a compound which is capable of inhibiting the processing of APP by γ-secretase as evidenced by in vitro assays such as those described later herein and in Biochemistry, (2000) 39(30), 8698-8704 and J. Neuroscience Methods (2000) 102, 61-68. Numerous examples of such compounds are known in the art, as described in Garofalo, Expert Opin. Ther. Patents (2008) 18(7), 693-703 and references therein, for example. Advantageously, but not necessarily, the GSI is a compound which inhibits processing of APP by γ-secretase to a greater extent than it inhibits processing of Notch by γ-secretase, as measured by in vitro assays. Suitable assays for inhibition of Notch processing are described later herein and in Biochemistry (2003) 42, 7580-7586. Assays which can measure inhibition of both Notch and APP processing simultaneously are disclosed in WO 2007/029030, which is hereby incorporated by reference in its entirety.

In one embodiment of the invention, the GSI is a compound of formula I:

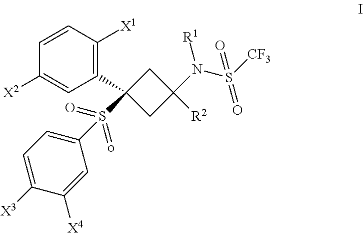

or pharmaceutically acceptable salt thereof, wherein:
X¹ is selected from the group consisting of: F and CN;
X² is selected from the group consisting of: F, Cl and CN;
X³ is selected from the group consisting of: F, Br, Cl, CN, CF₃, OCF₃, C(O)—OCH₃ and S—CH₃;
X⁴ is selected from the group consisting of: H, F and Cl;
R¹ is selected from the group consisting of:

(a) H,

(b) CH₃,

(c) —(CH₂)ₙ—OR³;

(d) —(CH₂)ₙ—C(O)—OR⁴ and

(e) —SO₂—CF₃;
R² is H or CH₃ when the compound of formula I is in the cis configuration, otherwise R² is H;
R³ is a five- or six-membered non-aromatic heterocycle having one oxygen heteroatom;
R⁴ is H or CH₃; and
n is 1 to 4.

In a sub-set of the compounds of formula I X¹ and X² are F; X³ is Cl; and X⁴ is H; and in a particular sub-embodiment the GSI is a compound of formula IA:

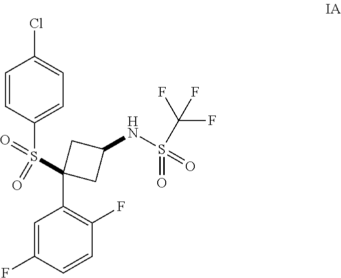

or a pharmaceutically acceptable salt thereof.

Compounds of formula I are described in WO 2009/131906, which is hereby incorporated by reference in its entirety.

In a second embodiment of the invention, the GSI is a compound of formula II:

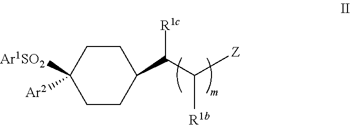

wherein:

m is 0 or 1;

Z represents CN, OR^{2a}, CO₂R^{2a} or CON(R^{2a})₂;

R^{1b} represents H, C₁₋₄alkyl or OH;

R^{1c} represents H or C₁₋₄alkyl;

Ar¹ represents phenyl or pyridyl, either of which bears 0-3 substituents independently selected from halogen, CN, NO₂, CF₃, OH, OCF₃, C₁₋₄alkoxy or C₁₋₄alkyl which optionally bears a substituent selected from halogen, CN, NO₂, CF₃, OH and C₁₋₄alkoxy;

Ar² represents phenyl which is substituted in the 2- and 5-positions with halogen;

R^{2a} represents H, C₁₋₆alkyl, C₃₋₆cycloalkyl, C₃₋₆cycloalkylC₁₋₆alkyl, C₂₋₆alkenyl, any of which optionally bears a substituent selected from halogen, CN, NO₂, CF₃, OR^{2b}, CO₂R^{2b}, N(R^{2b})₂, CON(R^{2b})₂, Ar and COAr; or R^{2a} represents Ar; or two R^{2a} groups together with a nitrogen atom to which they are mutually attached may complete an N-heterocyclyl group bearing 0-4 substituents independently selected from ═O, ═S, halogen, C₁₋₄alkyl, CN, NO₂, CF₃, OH, C₁₋₄alkoxy, C₁₋₄alkoxycarbonyl, CO₂H, amino, C₁₋₄alkylamino, di(C₁₋₄alkyl)amino, carbamoyl, Ar and COAr;

R^{2b} represents H, C₁₋₆alkyl, C₃₋₆cycloalkyl, C₃₋₆cycloalkylC₁₋₆alkyl, C₂₋₆alkenyl, any of which optionally bears a substituent selected from halogen, CN, NO₂, CF₃, OH, C₁₋₄alkoxy, C₁₋₄alkoxycarbonyl, CO₂H, amino, C₁₋₄alkylamino, di(C₁₋₄alkyl)amino, carbamoyl, Ar and COAr; or R^{2b} represents Ar; or two R^{2b} groups together with a nitrogen atom to which they are mutually attached may complete an N-heterocyclyl group bearing 0-4 substituents independently selected from ═O, ═S, halogen, C₁₋₄alkyl, CN, NO₂, CF₃, OH, C₁₋₄alkoxy, C₁₋₄alkoxycarbonyl, CO₂H, amino, C₁₋₄alkylamino, di(C₁₋₄alkyl)amino, carbamoyl, Ar and COAr;

Ar represents phenyl or heteroaryl bearing 0-3 substituents selected from halogen, C₁₋₄alkyl, CN, NO₂, CF₃, OH, C₁₋₄alkoxy, C₁₋₄alkoxycarbonyl, amino, C₁₋₄alkylamino, di(C₁₋₄alkyl)amino, carbamoyl, C₁₋₄alkylcarbamoyl and di(C₁₋₄alkyl)carbamoyl;

or a pharmaceutically acceptable salt thereof.

In a subset of the compounds of formula II Ar¹ represents 4-chlorophenyl or 4-trifluoromethylphenyl, Ar² represents 2,5-difluorophenyl, R^{1b} and R^{1c} are both H, and either m is 1 and Z represents CO₂R^{2a} or m is 0 and Z represents CON(R^{2a})₂. In a particular sub-embodiment the GSI is cis-4-[(4-chlorophenyl)sulfonyl]-4-(2,5-difluorophenyl)cyclohexanepropanoic acid or the sodium salt thereof, herein designated Compound IIA.

Compounds of formula II are described in WO 03/018543, U.S. Pat. No. 6,984,663 and U.S. Pat. No. 7,304,094, all of which are hereby incorporated by reference in their entirety. Methods for synthesizing compounds of formula II are described in WO 2004/013090 and U.S. Pat. No. 7,276,637, both of which are hereby incorporated by reference in their entirety.

In a third embodiment of the invention, the GSI is a compound of formula III:

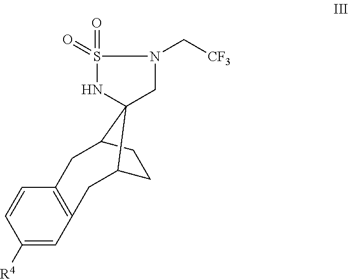

or a pharmaceutically acceptable salt thereof;

wherein R⁴ is —CH═CHCH₂N(R¹⁶)₂ where —N(R¹⁶)₂ is morpholin-4-yl, 4-trifluoromethylpiperidin-1-yl, 5-aza-2-oxabicyclo[2.2.1]hept-1-yl, 4,4-difluoropiperidin-1-yl, 4-hydroxy-4-trifluoromethylpiperidin-1-yl, 4-methylpiperidin-1-yl, 3-oxo-4-phenylpiperazin-1-yl, 3-oxo-4-cyclohexylpiperazin-1-yl, 3-oxo-piperazine-1-yl, N-(tetrahydrofuran-3-yl)amino, N-methyl-N-(tetrahydrofuran-3-yl)amino, N-(tetrahydropyran-4-yl)amino, N-methyl-N-(tetrahydropyran-4-yl)amino, N-(dioxanylmethyl)amino, N-[(tetrahydropyran-2-yl)methyl]amino, 3-hydroxypiperidin-1-yl, 5-aza-2-oxabicyclo[5.4.0]undeca-7,9,11-trien-5-yl, 2-(phenoxymethyl)morpholin-4-yl, N-[(4-phenylmorpholin-2-yl)methyl]amino, 3,3-difluoropyrrolidin-1-yl, N-(2,2,2-trifluoroethyl)amino, or 3-(pyridin-3-yl)pyrrolidin-1-yl.

In a particular sub-embodiment the GSI is the compound of formula III in which —N(R¹⁶)₂ is 4-trifluoromethylpiperidin-1-yl, or a pharmaceutically acceptable salt thereof, herein designated Compound IIIA.

Compounds of formula III are described in WO 02/36555, U.S. Pat. No. 7,138,400, and in Sparey et al, Bioorg. Med. Chem. Lett. (2005) 15, 4212-4216, all of which are hereby incorporated by reference in their entirety.

In a fourth embodiment of the invention, the GSI is a compound of formula IV:

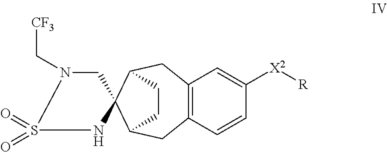

or a pharmaceutically acceptable salt thereof;

wherein X² is a bivalent pyrazole, imidazole, triazole, oxazole, isoxazole, thiazole, isothiazole, thiadiazole or 1,3,4-oxadiazole residue optionally bearing a hydrocarbon substituent comprising 1-5 carbon atoms which is optionally substituted with up to 3 halogen atoms; and R is selected from:

(i) CF₃ or an alkyl group of up to 6 carbon atoms, optionally substituted with halogen, CF₃, CHF₂, CN, OH, CO₂H, C₂₋₆acyl, C₁₋₄alkoxy or C₁₋₄alkoxycarbonyl;

(ii) a non-aromatic heterocyclic group comprising up to 7 ring atoms of which up to 3 are chosen from N, O and S and the remainder are carbon, bearing 0-3 substituents independently selected from oxo, halogen, CN, C₁₋₆alkyl, OH, CF₃, CHF₂, CH₂F, C₂₋₆acyl, CO₂H, C₁₋₄alkoxy and C₁₋₄alkoxycarbonyl;

(iii) phenyl or 6-membered heteroaryl, either of which bears 0-3 substituents independently selected from halogen, CF₃, CHF₂, CH₂F, NO₂, CN, OCF₃, C₁₋₆alkyl and C₁₋₆alkoxy; and

(iv) N(R^{a})₂ where each R^{a} independently represents H or C₁₋₆alkyl which is optionally substituted with halogen, CF₃, CHF₂, CN, OH, C₁₋₄alkoxy or C₁₋₄alkoxycarbonyl.

In a subset of the compounds of formula IV X² represents 5-substituted-thiazol-2-yl, 5-substituted-4-methylthiazol-2-yl, 5-substituted-1-methylpyrazol-3-yl, 1-substituted-imidazol-4-yl or 1-substituted-1,2,4-triazol-3-yl; and R represents 4-fluorophenyl, 4-chlorophenyl or 3,4-difluorophenyl. In a particular sub-embodiment the GSI is the compound of formula IV in which R—X²— represents 5-(4-fluorophenyl)-1-methylpyrazol-3-yl, herein designated Compound IVA.

Compounds of formula IV are described in WO 03/093252, U.S. Pat. No. 7,041,689, U.S. Pat. No. 7,282,513, WO 2004/039800 and U.S. Pat. No. 7,427,621, all of which are hereby incorporated by reference in their entirety.

In a fifth embodiment of the invention, the GSI is a compound of formula V:

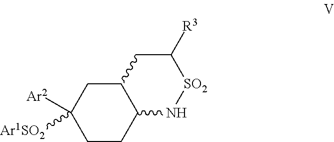

wherein the bonds indicated by wavy lines are mutually cis with respect to the cyclohexane ring;

R³ represents H or a hydrocarbon group of up to 10 carbon atoms, optionally substituted with CF₃, CHF₂, halogen, CN, OR⁵, COR⁵, CO₂R⁵, OCOR⁶, N(R⁵)₂, CON(R⁵)₂ or NR⁵COR⁶;

R⁵ represents H or C₁₋₄alkyl;

R⁶ represents C₁₋₄alkyl; and

Ar¹ and Ar² independently represent phenyl or heteroaryl, either of which bears 0-3 substituents independently selected from halogen, CN, NO₂, CF₃, CHF₂, OH, OCF₃, CHO, CH═NOH, C₁₋₄alkoxy, C₁₋₄alkoxycarbonyl, C₂₋₆acyl, C₂₋₆alkenyl and C₁₋₄alkyl which optionally bears a substituent selected from halogen, CN, NO₂, CF₃, OH and C₁₋₄alkoxy;

or a pharmaceutically acceptable salt thereof.

In a subset of the compounds of formula V, Ar¹ represents 4-chlorophenyl or 4-trifluoromethylphenyl, Ar² represents 2,5-difluorophenyl, and R³ represents H, C₁₋₆alkyl or C₂₋₆alkenyl. In a particular subembodiment, the GSI is selected from:
(4aRS,6RS,8aSR)-6-(2,5-difluorophenyl)-6-{[4-(trifluoromethyl)phenyl]sulfonyl}octahydro-1H-2,1-benzothiazine 2,2-dioxide;
(3R,4aS,6S,8aR)-6-(2,5-difluorophenyl)-3-ethyl-6-{[4-(trifluoromethyl)phenyl]sulfonyl}octahydro-1H-2,1-benzothiazine 2,2-dioxide;
(3S,4aS,6S,8aR)-6-(2,5-difluorophenyl)-3-ethyl-6-{[4-(trifluoromethyl)phenyl]sulfonyl}octahydro-1H-2,1-benzothiazine 2,2-dioxide;
(3RS,4aRS,6RS,8aSR)-6-(2,5-difluorophenyl)-3-isopropyl-6-{[4-(trifluoromethyl)phenyl]sulfonyl}octahydro-1H-2,1-benzothiazine 2,2-dioxide;
(3SR,4aRS,6RS,8aSR)-6-(2,5-difluorophenyl)-3-isopropyl-6-{[4-(trifluoromethyl)phenyl]sulfonyl}octahydro-1H-2,1-benzothiazine 2,2-dioxide; and
(3R,4aS,6S,8aR)-6-[(4-chlorophenyl)sulfonyl]-6-(2,5-difluorophenyl)-3-ethyloctahydro-1H-2,1-benzothiazine 2,2-dioxide;
and the pharmaceutically acceptable salts thereof;
more particularly (3R,4aS,6S,8aR)-6-(2,5-difluorophenyl)-3-ethyl-6-{[4-(trifluoromethyl)phenyl]sulfonyl}octahydro-1H-2,1-benzothiazine 2,2-dioxide, herein designated Compound VA.

Compounds of formula V are described in WO 2004/101539, U.S. Pat. No. 7,342,009, U.S. Pat. No. 7,410,964 and J. Org. Chem. (2006) 71(8), 3086-3092, all of which are hereby incorporated by reference in their entirety.

In a sixth embodiment of the invention the GSI is a compound of formula VIA or VIB:

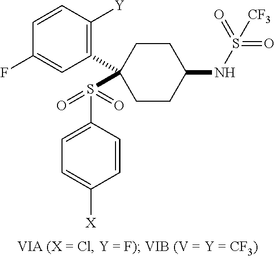

or a pharmaceutically acceptable salt thereof. Compound VIA is disclosed in U.S. Pat. No. 6,890,956, which is hereby incorporated by reference in its entirety, and compound. VIB may also be prepared by the methods disclosed therein.

Further examples of GSIs which may be used in the invention include semagacestat (LY-450139, WO 02/47671 and U.S. Pat. No. 7,468,365, both of which are hereby incorporated by reference in their entirety) and begacestat (U.S. Pat. No. 7,300,951, which is hereby incorporated by reference in its entirety).

The GSI is typically administered in the form of a pharmaceutical composition comprising the active ingredient and a pharmaceutically acceptable carrier. Preferably the composition is in unit dosage form such as tablets, pills, capsules, powders, granules, sterile parenteral solutions or suspensions, metered aerosol or liquid sprays, drops, ampoules, transdermal patches, auto-injector devices or suppositories; for oral, parenteral, intranasal, sublingual or rectal administration, or for administration by inhalation or insufflation. Most suitably, the GSI is administered orally in the form of tablets or capsules. The principal active ingredient typically is mixed with a pharmaceutical carrier, e.g. conventional tableting ingredients such as corn starch, lactose, sucrose, sorbitol, talc, stearic acid, magnesium stearate and dicalcium phosphate, or gums, dispersing agents, suspending agents or surfactants such as sorbitan monooleate and polyethylene glycol, and other pharmaceutical diluents, e.g. water, to form a homogeneous preformulation composition containing the GSI. When referring to these preformulation compositions as homogeneous, it is meant that the active ingredient is dispersed evenly throughout the composition so that the composition may be readily subdivided into equally effective unit dosage forms such as tablets, pills and capsules. This preformulation composition is then subdivided into unit dosage forms of the type described above containing a predetermined amount of the active ingredient, e.g. from 0.1 to about 500 mg of the active ingredient. Tablets or pills of the composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the compositions useful in the present invention may be incorporated for administration orally or by injection include aqueous solutions, liquid- or gel-filled capsules, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, poly(ethylene glycol), poly(vinylpyrrolidone) or gelatin.

Optimum dosage levels will vary according to a number of factors, notably the identity of the GSI, the particular intermittent regimen selected, the weight of the patient and the severity of the disease, and may be determined in each instance by protocols well-known to those skilled in the art. Generally speaking, for a given GSI the individual doses to be administered on an intermittent regimen in accordance with the invention will be greater than the individual doses that would be administered on a continuous regimen to give the equivalent therapeutic effect.

A specific example of a method in accordance with the invention is administration of a daily dose of 750-1500 mg of Compound IIA, in particular 1000 mg, on a cycle of 1 day on followed by 6 days off. A further example is administration of a daily dose of 250-500 mg of Compound IIA, in particular 350 mg, on a cycle of 3 days on followed by 4 days off.

Intermittent dosing of a GSI as taught herein may be combined with conventional administration of one or more additional compounds known to be useful in the treatment or prevention of AD or the symptoms thereof. Such additional compounds thus include cognition-enhancing drugs such as acetylcholinesterase inhibitors (e.g. donepezil and galanthamine), NMDA antagonists (e.g. memantine) or PDE4 inhibitors (e.g. Ariflo™ and the classes of compounds disclosed in WO 03/018579, WO 01/46151, WO 02/074726 and WO 02/098878, incorporated by reference in their entirety). Such additional compounds also include cholesterol-lowering drugs such as the statins, e.g. simvastatin and atorvastatin, and compounds which are reported to inhibit the aggregation of Aβ or otherwise attenuate its neurotoxicity, such as clioquinol (Gouras and Beal, Neuron, 30 (2001), 641-2), 3-aminopropane-1-sulfonic acid (also known as tramiprosate or Alzhemed™), phytic acid derivatives as disclosed in U.S. Pat. No. 4,847,082, incorporated by reference in its entirety, and inositol derivatives as taught in US 2004/0204387, incorporated by reference in its entirety.

### SYNTHESIS EXAMPLES

### Intermediate A

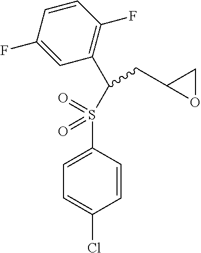

### 2-[1-[(4(4-chlorophenyl)sulfonyl]-2-(2,5-difluorophenyl)ethyl]oxirane

4-chlorophenyl-2,5-difluorobenzylsulfone was prepared as described in WO 02/081435, U.S. Pat. No. 7,595,344 and U.S. Pat. No. 7,598,386, incorporated by reference in their entirety (Intermediate 1) from 4-chlorothiophenol and 2,5-difluorobenzyl bromide in two steps.

4-chlorophenyl-2,5-difluorobenzylsulfone (12 g, 39.6 mmol) in THF (99 ml) was treated with ⁿBuLi (19 ml, 2.5 M in hexane, 47.6 mmol) at 0° C. for 10 min followed by addition of epichlorohydrin (3.73 ml, 47.6 mmol). The reaction was slowly warmed to room temperature for 14 h, quenched with water (100 ml) and diluted with EtOAc (300 ml). The organic phase was separated, dried (Na₂SO₄) and evaporated to dryness to give an oil. This material was chromatographed on silica, eluting with 10-45% ethyl acetate in hexanes to afford 9.8 g of the desired product as off-white solid. ¹H NMR (600 MHZ, CDCl₃) two diastereomers (˜1/1) δ 7.52 (m,2H/2H), 7.38 (m,2H/2H), 7.30-7.22 (m, 1H/1H), 6.99 (m, 1H/1H), 6.86-6.80 (m, 1H/1H), 4.76-4.70 (m, 1H/1H), 3.03-2.48 (m, 4H/4H), 2.37 (m, 1H), 2.20 (m, 1H). MS calculated 359.0 (MH⁺), exp 358.9 (MH⁺).

### Intermediate B

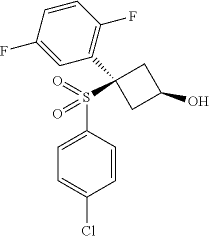

### cis-3-[(4-chlorophenyl)sulfonyl]-3-(2,5-difluorophenyl)cyclobutanol

To 2-[2-[(4-chlorophenyl)sulfonyl]-2-(2,5-difluorophenyl)ethyl]oxirane (100 mg, 0.279 mmol) in THF (2.7 ml) was added MeMgBr (279 μL, 3M in ether, 0.836 mmol) at −78° C. The reaction was warmed to room temperature over 1 h then quenched with sat. NH₄Cl (3 ml) and diluted with EtOAc (10 ml). The organic phase was washed with brine (10 ml), dried (Na₂SO₄) and evaporated to dryness to afford the desired product (100 mg). ¹H NMR (600 MHZ, CDCl₃) δ 7.66 (d, J=8.4 Hz, 2H), 7.57 (d, J=8.4 Hz, 2H), 6.99 (m, 1H), 6.82 (m, 1H), 6.74 (m, 1H), 4.28 (m, 1H), 3.47 (d, J=11.4 Hz, 1H, OH), 3.13 (m, 4H). MS calculated 422.0 (MNa⁺+CH₃CN), exp 421.9 (MNa⁺+CH₃CN).

### Intermediate C

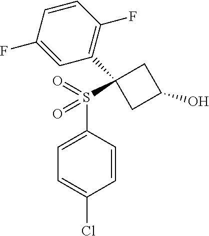

### trans-3-[(4-chlorophenyl)sulfonyl]-3-(2,5-difluorophenyl)cyclobutanol

To PPh₃ (8.19 g, 31.2 mmol) in THF (100 ml) was added DIAD (6.31 g, 31.2 mmol); the resulting mixture was stirred at room temperature for 0.5 h. The mixture was cooled to −50° C. and cis-3-[(4-chlorophenyl)sulfonyl]-3-(2,5-difluorophenyl)cyclobutanol (8 g, 22.3 mmol) in THF (10 ml) was added. The reaction was stirred for 20 min followed by addition of solid 4-nitrobenzoic acid (5.22 g, 31.2 mmol). The resulting mixture was warmed to room temperature and allowed to stir at room temperature for 20 h. The reaction was then cooled to 0° C. to which was added NaOMe (134 ml, 0.5 M in MeOH, 66.9 mmol). After 40 min the reaction was quenched with Sat. NH₄Cl (100 ml) and diluted with EtOAc (300 ml). The organic phase was separated, dried (Na₂SO₄) and evaporated to dryness to afford an oil. This material was chromatographed on silica, eluting with ether in hexanes to give 8 g of the title product as white solid containing ˜15% of cis-3-[(4-chlorophenyl)sulfonyl]-3-(2,5-difluorophenyl)cyclobutanol (starting material). ¹H NMR (600 MHZ, CDCl₃) major product, δ 7.35 (m, 4H), 6.96 (m, 1H), 6.80-6.72 (m, 2H), 4.84 (m, 1H), 3.49 (m, 2H), 2.59 (m, 2H). MS calculated 422.0 (MNa⁺+CH₃CN), exp 421.9 (MNa⁺+CH₃CN).

### Intermediate D

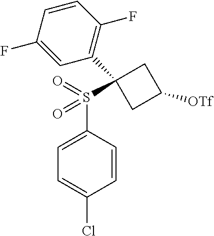

### trans-3-[(4-chlorophenyl)sulfonyl]-3-(2,5-difluorophenyl)cyclobutyl trifluoromethanesulfonate

To trans-3-[(4-chlorophenyl)sulfonyl]-3-(2,5-difluorophenyl)cyclobutanol (2 g, 5 mmol) in DCM (27.9 ml) was added trifluoromethanesulfonic anhydride (1.13 ml, 6.69 mmol) and pyridine (0.902 ml, 11.15 mmol) at 0° C. The reaction was stirred for 30 min, quenched with Sat. NH₄Cl (30 ml) and diluted with EtOAc (150 ml). The organic phase was separated, dried (Na₂SO₄) and evaporated to dryness to give an oil. This material was chromatographed on silica, eluting with 0-25% ethyl acetate in hexanes to afford 2.7 g of the desired product as white solid, ¹H NMR (600 MHZ, CDCl₃) δ 7.36 (m, 4H), 7.02 (m, 1H), 6.79 (m, 2H), 5.72 (m, 1H), 3.66 (broad s, 2H), 3.02 (dd, =14.4, 6.6 Hz, 2H). MS calculated 554.0 (MNa⁺+CH₃CN), exp 553.8 (MNa⁺+CH₃CN).

### Intermediate E

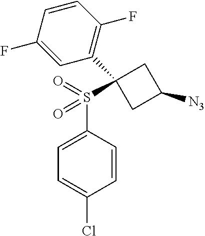

### cis-3-azido-1-(2,5-difluorophenyl)cyclobutyl 4-chlorophenyl sulfone

A mixture of trans-3[(4-chlorophenyl)sulfonyl]-3-(2,5-difluorophenyl)cyclobutyl trifluoromethanesulfonate (3.9 g, 7.95 mmol) and sodium azide (5.17 g, 79 mmol) in ethanol (39.7 ml) and water (39.7 ml) was heated at 85° C. for 2 h. The mixture was cooled to room temperature and diluted with water (100 ml) and EtOAc (150 ml). The organic phase was separated, dried (Na₂SO₄) and evaporated to dryness to afford an oil. This material was chromatographed on silica, eluting with 0-30% ethyl acetate in hexanes to afford the desired 2.3 g product as white solid. ¹H NMR (600 MHZ, CDCl₃) δ 7.35 (m, 4H), 7.02 (m, 1H), 6.91 (m, 1H), 6.80 (m, 1H), 3.81 (m, 1H), 3.30 (m, 2H), 3.02 (m, 2H).

### Intermediate F

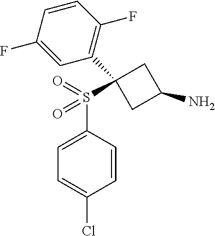

### cis-3-[(4-chlorophenyl)sulfonyl]-3-(2,5-difluorophenyl)cyclobutanamine

cis-3-Azido-1-(2,5-difluorophenyl)cyclobutyl 4-chlorophenyl sulfone (2.07 g, 5.39 mmol) and palladium (0.861 g, 10% on carbon, 0.809 mmol) in MeOH (27 ml) was stirred under H₂ balloon for 4 h. The crude mixture was filtered through a silica gel pack and washed with 5:1 DCM/MeOH (100 ml) to remove palladium residue. The solvent was removed to give the product which was used directly in next transformation without further purification. ¹H NMR (600 MHZ, CDCl₃) δ 7.34 (m, 4H), 6.97 (m, 1H), 6.84 (m, 1H), 6.77 (m, 1H), 3.45 (m, 1H), 3.05-2,94 (m, 4H). MS calculated 358.0 (MH⁺), exp 358.0 (MH⁺).

### Method (b)

To a solution of cis-3-azido-1-(2,5-difluorophenyl)cyclobutyl 4-chlorophenyl sulfone (9.5 g, 24.75 mmol) in ethanol/THF stirred at RT was added zinc (3.24 g, 49.5 mmol), followed by ammonium formate (3.12 g, 49.5 mmol). Reaction was stirred at room temperature for 1 h. The mixture was filtered through Celite and ther solvents were removed. To the resulting residue was added 100 ml of sat'd NaHCO3 solution and the products was extracted with EtOAc (2×100 ml). Combined organics were washed with brine and dried over anhydrous sodium sulfate, and filtered through Celite. The filtrate was concentrated to afford the desired product

### Intermediate G

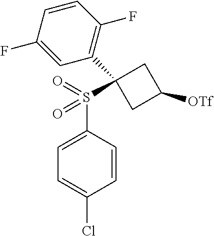

### cis-3-[(4-chlorophenyl)sulfonyl]-3-(2,5-difluorophenyl)cyclobutyl trifluoromethanesulfonate

Prepared as for trans-3-[(4-chlorophenyl)sulfonyl]-3-(2,5-difluorophenyl)cyclobutyl trifluoromethanesulfonate, using cis-3-[(4-chlorophenyl)sulfonyl]-3-(2,5-difluorophenyl)cyclobutanol:

¹H NMR (600 MHZ, CDCl₃) δ 7.35 (m, 4H), 7.04 (m, 1H), 6.88 (m, 1H), 6.81 (m, 1H), 5.08 (m, 1H), 3.60 (m, 2H), 3.23 (m, 2H). MS calculated 554.0 (MNa⁺+CH₃CN), exp 553.8 (MNa⁺+CH₃CN).

### Intermediate H

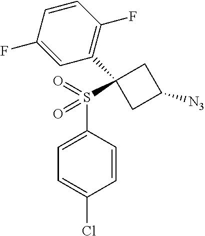

### trans-3-azido-1-(2,5-difluorophenyl)cyclobutyl 4-chlorophenyl sulfone

Prepared as for cis-3-azido-1-(2,5-difluorophenyl)cyclobutyl 4-chlorophenyl sulfone, using cis-3-[(4-chlorophenyl)sulfonyl]-3-(2,5-difluorophenyl)cyclobutyl trifluoromethanesulfonate.

¹H NMR (600 MHZ, CDCl₃) δ 7.35 (m, 4H), 6.98 (m, 1H), 6.76 (m, 2H), 4.56 (m, 1H), 3.49 (m, 2H), 2,66 (m, 2H).

### Intermediate I

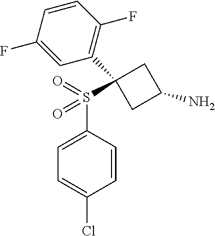

### trans-3-[(4-chlorophenyl)sulfonyl]-3-(2,5-difluorophenyl)cyclobutanamine

Prepared as for cis-3-[(4-chlorophenyl)sulfonyl]-3-(2,5-difluorophenyl)cyclobutanamine, using trans-3-azido-1-(2,5-difluorophenyl)cyclobutyl 4-chlorophenyl sulfone.

¹H NMR (600 MHZ, CD₃OD) δ 7.48-7.41 (m, 4H), 7.07 (m, 1H), 6.88 (m, 1H), 6.79 (m, 1H), 3.88 (m, 1H), 3.41 (m, 2H), 2.50 (m, 2H). MS calculated 358.0 (MH⁺), exp 358.0 (MH⁺).

### Intermediate J

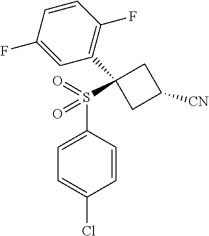

### trans-3-[(4-chlorophenyl)sulfonyl]-3-(2,5-difluorophenyl)cyclobutanecarbonitrile

cis-3-[(4-chlorophenyl)sulfonyl]-3-(2,5-difluorophenyl)cyclobutyl trifluoromethanesulfonate (4 g, 8.15 mmol) and tetrabutylammonium cyanide (5.47 g, 20.37 mmol) in DMSO (54 ml) was heated at 80° C. for 45 min. The resulting mixture was cooled to room temperature and diluted with water (200 ml) and EtOAc (250 ml). The organic phase was washed with water, brine, separated, dried (Na₂SO₄) and evaporated to dryness, to afford an oil. This material was chromatographed on silica, eluting with 0-50% ethyl acetate in hexanes to give the desired product (2.9 g) as off-white solid. ¹H NMR (600 MHZ, CDCl₃) δ 7.38-7.33 (m, 4H), 7.02 (m, 1H), 6.80-6.74 (m, 2H), 3.74 (m, 1H), 3.51 (m, 2H), 3.02 (m, 2H). MS calculated 431.0 (MNa⁺+CH₃CN), exp 431.0 (MNa⁺+CH₃CN).

### Intermediate K

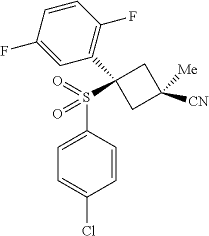

### cis-3-[(4-chlorophenyl)sulfonyl]-3-(2,5-difluorophenyl)-1-methylcyclobutanecarbonitrile

To trans-3-[(4-chlorophenyl)sulfonyl]-3-(2,5-difluorophenyl)cyclobutanecarbonitrile (600 mg, 1.63 mmol) in THF (8 mL) was added LiHMDS (2.45 ml, 1 M in THF, 2.45 mmol) at −78° C. After 10 min MeI (306 μl, 4.89 mmol) was introduced to reaction mixture. The reaction was stirred for 2 h with the temperature slowly increasing to 0° C. The reaction was then quenched with water and extracted with EtOAc. The organic phase was separated, dried (Na₂SO₄) and evaporated to dryness to afford an oil. This material was chromatographed on silica, eluting with 0-50% ethyl acetate in hexanes to give the product (260 mg) as a single diastereomeric products. ¹H NMR (600 MHZ, CD₃OD) δ 7.36 (s, 4H), 7.02 (m, 1H), 6.85-6.78 (m, 2H), 3.79 (d, J=14.4 Hz, 2H), 2.72 (d, J=14.4 Hz, 2H), 1.44 (s, 3H). MS calculated 785.1 (2M+Na⁺), exp 785.0 (2M+Na⁺).

### Intermediate L

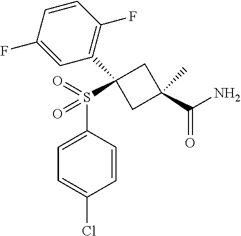

### cis-3-[(4-chlorophenyl)sulfonyl]-3-(2,5-difluorophenyl)-1-methylcyclobutanecarboxamide

To cis-3-[(4-chlorophenyl)sulfonyl]-3-(2,5-difluorophenyl)-1-methylcyclobutanecarbonitrile (500 mg, 1.31 mmol) and K₂CO₃ (362 mg, 2.62 mmol) in DMSO (6.5 ml) was added H₂O₂ (1.15 ml, 35% in water, 13.1 mmol) dropwise and the reaction was stirred vigorously for 2 h. The mixture was diluted with water (50 ml) and EtOAc (50 ml). The organic phase was washed with water, brine, separated, dried (Na₂SO₄) and evaporated to dryness to give cis-3-[(4-chlorophenyl)sulfonyl]-3-(2,5-difluorophenyl)-1-methylcyclobutanecarboxamide (500 mg) as off-white solid which was used in the next reaction without further purification. ¹H NMR (600 MHZ, CDCl₃) δ 7.35 (m, 4H), 7.02 (m, 1H), 6.87-6.79 (m, 2H), 6.54 (broad s, 1H), 6.40 (broad s, 1H), 3.67 (d, J=14.4 Hz, 2H), 2.62 (d, J=14.4 Hz, 2H), 1.28 (s, 3H). MS calculated 400.0 (MH⁺), exp 400.0 (MH⁺).

### Intermediate M

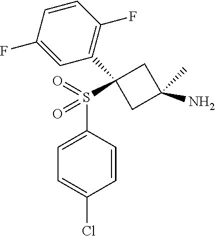

### cis-3-[(4-chlorophenyl)sulfonyl]-3-(2,5-difluorophenyl)-1-methylcyclobutanamine

cis-3-[(4-chlorophenyl)sulfonyl]-3-(2,5-difluorophenyl)-1-methylcyclobutanecarboxamide (400 mg, 1 mmol) and PIFA (473 mg, 1.1 mmol) in acetonitrile (2.5 ml) and water (2.5 ml) was stirred at 0° C. and the mixture was slowly warmed up to room temperature over 27 h. The reaction was then quenched with Sat. NaHCO₃ and extracted with EtOAc. The organic phase was separated, dried (Na₂SO₄) and evaporated to dryness to give an oil. This material was chromatographed on silica, eluting with 0-40% MeOH in DCM to give cis-3-[(4-chlorophenyl)sulfonyl]-3-(2,5-difluorophenyl)-1-methylcyclobutanamine (380 mg) as product. ¹H NMR (600 MHZ, CD₃OD) δ 7.41-7.36 (m, 4H), 7.02 (m, 1H), 6.81 (m, 1H), 6.74 (m, 1H), 3.58 (d, J=15.6 Hz, 2H), 2.89 (d, J=15.6 Hz, 2H), 1.58 (s, 3H). MS calculated 372.1 (MH⁺), exp 372.0 (MH⁺).

### Intermediate N

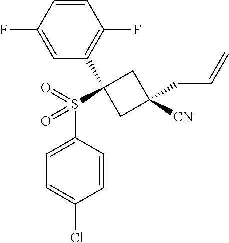

### cis-1-allyl-3-[(4-chlorophenyl)sulfonyl]-3-(2,5-difluorophenyl)cyclobutanecarbonitrile

Prepared as for cis-3-[(4-chlorophenyl)sulfonyl]-3-(2,5-difluorophenyl)-1-methylcyclobutanecarbonitrile, using trans-3-[(4-chlorophenyl)sulfonyl]-3-(2,5-difluorophenyl)cyclobutanecarbonitrile and allyl bromide.

¹H NMR (600 MHZ, CDCl₃) δ 7.36 (s, 4H), 7.03 (m, 1H), 6.83-6.78 (m, 2H), 5.70 (m, 1H), 5.18 (dd, J=10.2, 1.2 Hz, 1H), 5.07 (dd, 1H, J=16.8, 1.2 Hz, 1H), 3.70 (d, J=15.0 Hz, 2H), 2.77 (d, J=15.0 Hz, 2H), 2.32 (d, J=6.6 Hz, 2H). MS calculated 837.1 (2M+Na⁺), exp 837.0 (2M+Na⁺).

### Intermediate O

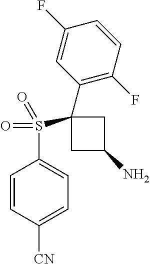

### 4-{[cis-3-amino-1-(2,5-difluorophenyl)cyclobutyl]sulfonyl}benzonitrile

Zinc cyanide (0.295 g, 2.52 mmol), Pd₂(dba)₃ (0.384 g, 0.419 mmol), Zinc (0.030 g, 0.461 mmol), DPPF (0.465 g, 0.838 mmol) and cis-3-[(4-chlorophenyl)sulfonyl]-3-(2,5-difluorophenyl)cyclobutanamine (1.5 g, 4.19 mmol) were taken up in DMA and stirred in a 25 mL Schlenk tube under an argon environment at 135° C. for 16 hours. Water was then added and the mixture was extracted with EtOAc. The organic layer was washed with a saturated NaHCO₃ solution and brine then dried over MgSO₄, filtered and concentrated. The residue was purified via silica column chromatography (0→8% MeOH/DCM) to give the title compound. MS: cal'd 349 (MH+), exp 349 (MH+)

### Example 1

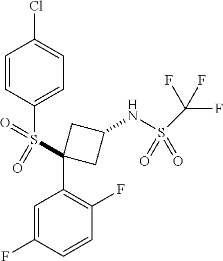

### N-[trans-3-[(4-chlorophenyl)sulfonyl]-3-(2,5-difluorophenyl)cyclobutyl]-1,1,1-trifluoromethanesulfonamide

To cis-3-[(4-chlorophenyl)sulfonyl]-3-(2,5-difluorophenyl)cyclobutanamine (1.5 g, 4.19 mmol) in DCM (27.9 ml) was added triethylamine (1.169 ml, 8.38 mmol) and trifluoromethanesulfonic anhydride (0.850 ml, 5.03 mmol) at 0° C. and the mixture was stirred for 2 h.

The mixture diluted with water (50 ml) and EtOAc (100 ml). The organic phase was washed with brine, separated, dried (Na₂SO₄) and evaporated to dryness to afford an oil. This material was chromatographed on silica, eluting with ethyl acetate in hexanes to give the desired product (1.45 g as white solid). ¹H NMR (600 MHZ, CDCl₃) δ 7.39 (d, J=9.0 Hz, 2H), 7.34 (d, J=9.0 Hz, 2H), 7.02 (m, 1H), 6.77 (m, 2H), 4.22 (m, 1H), 3.23 (m, 4H). MS calculated 553.0 (MNa⁺+CH₃CN), exp 553.0 (MNa⁺+CH₃CN).

### Example 2

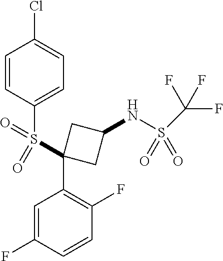

### N-[cis-3-[(4-chlorophenyl)sulfonyl]-3-(2,5-difluorophenyl)cyclobutyl]-1,1,1-trifluoromethanesulfonamide

Prepared as for Example 1, using trans-3-[(4-chlorophenyl)sulfonyl]-3-(2,5-difluorophenyl)cyclobutanamine.

¹H NMR (600 MHZ, CDCl₃) δ 7.33 (m, 4H), 6.98 (m, 1H), 6.74 (m, 2H), 5.35 (d, J=7.8 Hz, 1H, NH), 4.67 (m, 1H), 3.59 (m, 2H), 2.68 (m, 2H). MS calculated 553.0 (MNa⁺+CH₃CN), exp 552.8 (MNa⁺+CH₃CN).

The following were prepared by similar procedures:

| | | | | Salt |
|---|---|---|---|---|
| # | Structure | Name | MS | form |
| 3 | 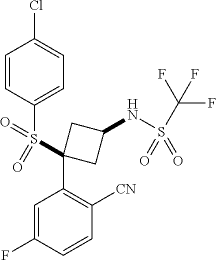 | N-[cis-3-[(4-chlorophenyl)sulfonyl]-3-(2- cyano-5-fluorophenyl)cyclobutyl]-1,1,1- trifluoromethanesulfonamide | Cal'd 519.0 (MNa+), exp 519.0 | Free base |
| 4 | 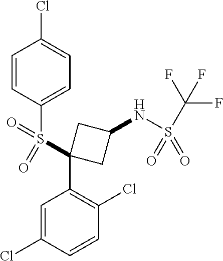 | N-[cis-3-[(4-chlorophenyl)sulfonyl]-3- (2,5-dichlorophenyl)cyclobutyl]-1,1,1- trifluoromethanesulfonamide | Cal'd 543.9 (MNa+), exp 543.9. | Free base |
| 5 | 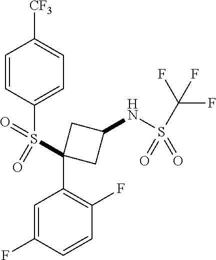 | N-(cis-3-(2,5-difluorophenyl)-3-{[4- (trifluoromethyl)phenyl]sulfonyl} cyclobutyl)-1,1,1- trifluoromethanesulfonamide | Cal'd 546 (MNa+), exp 546. | Free base |

### Example 6

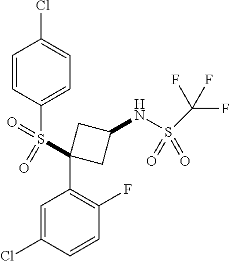

### N-{cis-3-(5-chloro-2-fluorophenyl)-3-[(4-chlorophenyl)sulfonyl]cyclobutyl}-1,1,1-trifluoromethanesulfonamide

MS Cal'd 506 (MNa+), exp 529; ¹H NMR (400 MHz, CDCl₃) δ 7.41˜7.43 (d, J=8.8 Hz, 2H), 7.34˜7.36 (d, J=8.4 Hz, 2H), 7.28˜7.33 (m, 1H), 6.98˜7.01 (m, 1H), 6.75˜6.83 (m, 2H), 4.20˜4.29 (m, 1H), 3.20˜3.32 (m, 4H).

### Example 7

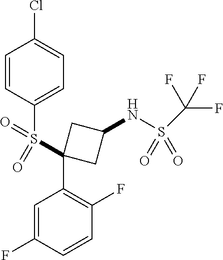

### N-{cis-3-(2,5-difluorophenyl)-3-[(4-fluorophenyl)sulfonyl]cyclobutyl}-1,1,1-trifluoromethanesulfonamide

¹H NMR (400 MHz, CDCl₃): δ 7.45˜7.49 (m, 2H), 7.12˜7.29 (m, 2H), 7.04˜7.10 (m, 1H), 6.80˜6.85 (m, 3H), 4.24˜4.30 (m, 1H), 3.24˜3.35 (m, 4H).

### Example 8

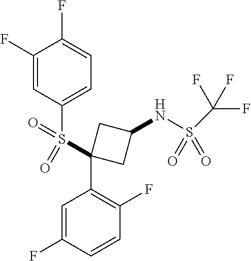

### N-{cis-3-(2,5-difluorophenyl)-3-[(3,4-difluorophenyl)sulfonyl]cyclobutyl}-1,1,1-trifluoromethanesulfonamide

MS Cal'd 514 (MH+), exp 514; ¹H NMR (400 MHz, CDCl₃): δ 7.15˜7.19 (m, 3H), 6.97˜7.04 (m, 1H), 6.72˜6.80 (m, 2H), 6.63˜6.65 (d, J=10.4 Hz, 1H), 4.13˜4.23 (m, 1H), 3.14˜3.26 (m, 4H).

### Example 9

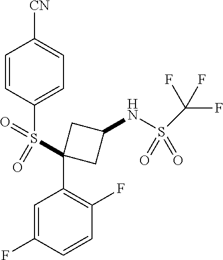

### N-[cis-3-[(4-cyanophenyl)sulfonyl]-3-(2,5-difluorophenyl)cyclobutyl]-1,1,1-trifluoromethanesulfonamide

MS: cal'd 502 (MNa+), exp 502 (MNa+). ¹H NMR (CDCl₃ 600 MHz) 7.67 (d, J=4.1 Hz, 2H), 7.58 (d, J=4.1, 2H), 7.08 (bm, 1H), 6.84 (m, 1H), 6.68 (m, 1H), 6.60 (d, 1H), 4.24 (bm, 1H), 3.4-3.2 (bm, 4H)

### Intermediate P

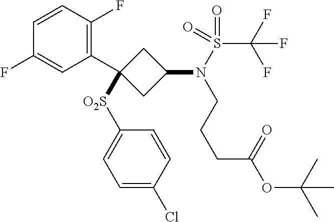

### Tert-Butyl 4-{[3-[(4-chlorophenyl)sulfonyl]-3-(2,5 difluorophenyl)cyclobutyl][(trifluoromethyl)sulfonyl]amino}butanoate

N-[cis-3-[(4-chlorophenyl)sulfonyl]-3-(2,5-difluorophenyl)cyclobutyl]-1,1,1-trifluoromethanesulfonamide (190 mg, 0.388 mmol) was added to DMF (1.1 mL) and treated with potassium carbonate (59 mg, 0.427 mmol), tert-butyl 4-bromobutanoate (95 mg, 0.427 mmol). The mixture was heated to 80° C. and stirred for 16 hours. The reaction was cooled to ambient temperature, diluted with ethyl acetate, and washed with 1/2 saturated brine solution twice. The organic layer was dried over anhydrous magnesium sulfate, filtered then concentrated in vacuo. The residue was purified by MPLC (0-30% EtOAc:Hept) to give the title compound. MS: cal'd 654 (M Na+), exp 654 (M Na+)

### Example 10

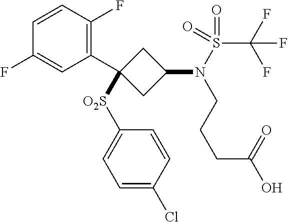

### 4-{[cis-3-[(4-chlorophenyl)sulfonyl]-3-(2,5-difluorophenyl)cyclobutyl][trifluoromethyl)sulfonyl]amino}butanoic acid

Tert-Butyl 4-{[3-[(4-chlorophenyl)sulfonyl]-3-(2,5 difluorophenyl)cyclobutyl][(trifluoromethyl)sulfonyl]amino}butanoate (144 mg, 0.228 mmol) was added to 1:1 DCM:TFA (1.1 mL) and stirred at ambient temperature for 40 minutes. The reaction mixture was concentrated in vacuo. The title compound was isolated as a white solid after trituration with heptane. ¹H NMR (DMSO D₆, 600 MHz) [symbol] 12.25 (s, 1H), 7.58 (d, J=8.2 Hz, 2H), 7.43 (d, J=8.2 Hz, 2H), 7.25-7.31 (m, 1H), 7.07-7.16 (m, 2H), 4.18-4.28 (m, 1H), 3.53 (s br, 2H), 3.30-3.42 (m, 2H), 3.08 (s br, 2H), 2.26-2.38 (m, 2H), 1.80-1.90 (m, 2H). MS: cal'd 598 (M Na+), exp 598 (M Na+).

### Example 11

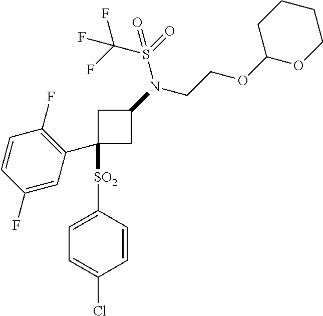

### N-[cis-3-[(4-chlorophenyl)sulfonyl]-3-(2,5-difluorophenyl)cyclobutyl]-1,1,1-trifluoro-N-[2-(tetrahydro-2-pyran-2-yloxy)ethyl]methanesulfonamide

N-[cis-3-[(4-chlorophenyl)sulfonyl]-3-(2,5-difluorophenyl)cyclobutyl]-1,1,1-trifluoromethanesulfonamide (50 mg, 0.102 mmol) was dissolved in anhydrous DMF (0.3 mL) and to this stirring solution was added potassium carbonate (49 mg, 0.357 mmol) followed by 2-(2-bromoethoxy)tetrahydro-2H-pyran (53 mg, 0.255 mmol). The resulting mixture was stirred at 80° C. for 16 hours. The mixture was cooled to ambient temperature, diluted with water, and extracted with EtOAc. The organic layer was again washed with saturated aqueous bicarbonate solution, then dried over anhydrous magnesium sulfate and concentrated in vacuo. The extract was purified by MPLC (0-45% EtOAc/DCM) to give the title compound. Rf=0.71 in 40% EtOAc/DCM. ¹H NMR (CDCl₃, 600 MHz) [symbol] 7.31-7.38 (m, 4H), 7.00-7.07 (m, 2H), 6.88-6.93 (m, 1H), 6.78-6.84 (m, 1H), 4.65-4.68 (m, 1H), 4.06-4.08 (m, 1H), 3.50-4.00 (m, 7H), 2.80-3.10 (m, 2H), 1.20-1.90 (m, 7H). MS: cal'd 640 (M Na+), exp 640 (M Na+)

### Example 12

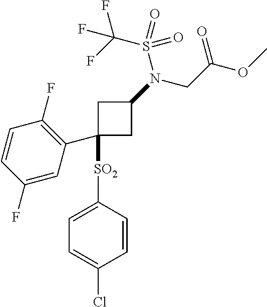

### Methyl{[cis-3-[(4-chlorophenyl)sulfonyl]-3-(2,5-difluorophenyl)cyclobutyl][(trifluoromethyl)sulfonyl]amino}acetate

Anhydrous THF (0.8 mL) was added to N-[cis-3-[(4-chlorophenyl)sulfonyl]-3-(2,5-difluorophenyl)cyclobutyl]-1,1,1-trifluoromethanesulfonamide (200 mg, 0.408 mmol) and the reaction was then cooled to 0° C. Sodium hydride (49 mg, 1.225 mmol) was added in one portion and the mixture was stirred at 0° C. for 15 minutes. The mixture effervesced and stirred as an off white suspension. After 15 minutes methyl bromoacetate (187 mg, 1.225 mmol) was added and the mixture effervesced again as it turned yellow. The mixture was stirred for 16 hours then quenched with saturated aq ammonium chloride and extracted with EtOAc. The organic layer was again washed with saturated aq ammonium chloride then dried over anhydrous magnesium sulfate and concentrated in vacuo. The extract was purified by MPLC (0-45% EtOAc:Hept) to give the title compound. Rf=0.6 in 40% EtOAc:Hept. ¹H NMR (CDCl₃, 600 MHz) [symbol] 7.36 (d, J=7.0 Hz, 2H), 7.43 (d, J=7.0 Hz, 2H), 7.02-7.08 (m, 1H), 6.86-6.90 (m, 1H), 6.78-6.84 (m, 1H), 4.30-4.60 (m, 3H), 3.85 (s, 3H), 3.38-3.34 (m, 2H), 3.04 (s br, 2H). MS: cal'd 584 (M Na+), exp 584 (M Na+)

The following list of compounds was prepared by similar procedures:

| | | | | Salt |
|---|---|---|---|---|
| # | Structure | Name | MS | form |
| 13 | 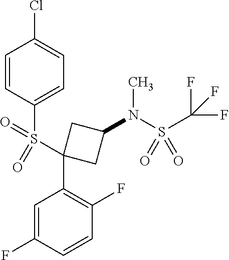 | N-[3-[(4-chlorophenyl)sulfonyl]-3-(2,5- difluorophenyl)cyclobutyl]-1,1,1- trifluoro-N-methylmethanesulfonamide | Cal'd 526.0 (MNa+), exp 525.8 (MNa+) | Free base |
| 14 | 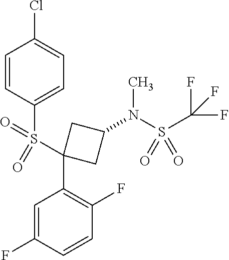 | N-[3-[(4-chlorophenyl)sulfonyl]-3-(2,5- difluorophenyl)cyclobutyl]-1,1,1- trifluoro-N-methylmethanesulfonamide | Cal'd 567.0 (MNa⁺ + MeCN), exp 566.8 (MNa⁺ + MeCN). | Free base |
| 15 | 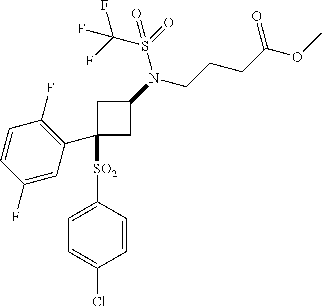 | methyl 4-{[cis-3-[(4- chlorophenyl)sulfonyl]-3-(2,5- difluorophenyl)cyclobutyl][(trifluoro- methyl)sulfonyl]amino}butanoate | Cal'd 612.0 (MNa+), exp 612.0 | Free base |
| 16 | 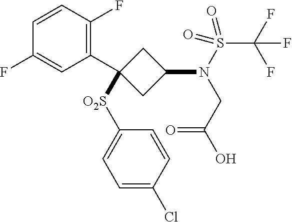 | N-[cis-3-[(4-chlorophenyl)sulfonyl]-3- (2,5-difluorophenyl)cyclobutyl]-N- [(trifluoromethyl)sulfonyl]glycine | Cal'd 570 (MNa+), exp 570. | Free base |

### Example 17

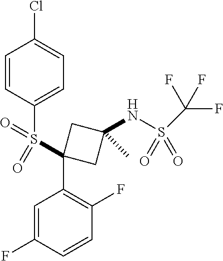

### N-[cis-3-[(4-chlorophenyl)sulfonyl]-3-(2,5-difluorophenyl)-1-methylcyclobutyl]-1,1,1-trifluoromethanesulfonamide

Prepared as for Example 1, using cis-3-[(4-chlorophenyl)sulfonyl]-3-(2,5-difluorophenyl)-1-methylcyclobutanamine.

¹H NMR (600 MHZ, CDCl₃) δ 7.33 (m, 4H), 7.00 (m, 1H), 6.90 (s, 1H, NH), 6.82-6.75 (m, 2H), 3.54 (d, J=14.4 Hz, 2H), 2.87 (d, J=14.4 Hz, 2H), 1.44 (s, 3H). MS calculated 526.0 (MNa⁺), exp 525.9 (MNa⁺).

### Example 18

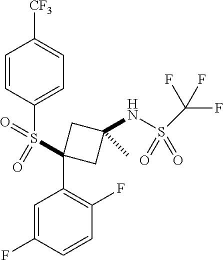

### N-(cis-3-(2,5-difluorophenyl)-1-methyl-3-{[4-(trifluoromethyl)phenyl]sulfonyl}cyclobutyl)-1,1,1-trifluoromethanesulfonamide

Prepared using procedures similar to example 17. Calcd (2M+Na)+: 1097.0, Found: 1096.5.

### Example 19

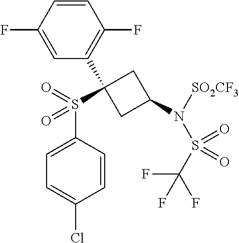

### N-[cis-3-[(4-chlorophenyl)sulfonyl]-3-(2,5-difluorophenyl)cyclobutyl]-1,1,1-trifluoro-N-[(trifluoromethyl)sulfonyl]methanesulfonamide

Prepared in Example 1 synthesis as byproduct.

¹H NMR (600 MHZ, CDCl₃) δ 7.38-7.33 (m, 4H), 7.07 (m, 1H), 6.95 (m, 1H), 6.84 (m, 1H), 4,44 (m, 1H), 3.82 (m, 2H), 315 (m, 2H). MS calculated 684.9 (MNa⁺+CH₃CN), exp 684.9 (MNa⁺+CH₃CN).

### Example 20

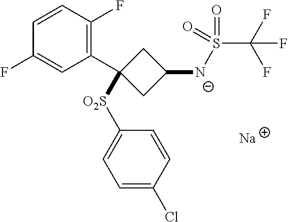

### Sodium[cis-3-[(4-chlorophenyl)sulfonyl]-3-(2,5-difluorophenyl)cyclobutyl][(trifluoromethyl)sulfonyl]azanide

Sodium hydride was suspended in hexane and cooled to 0° C. N-[cis-3-[(4-chlorophenyl)sulfonyl]-3-(2,5-difluorophenyl)cyclobutyl]-1,1,1-trifluoromethanesulfonamide (200 mg, 0.408 mmol) in THF (1 mL) was added dropwise to the sodium hydride suspension. The resulting mixture was stirred at 0° C. for 15 minutes then at ambient temperature for 30 minutes. At which time, the reaction mixture was concentrated in vacuo. A dry white powder was scraped out of the flask, placed in a glass fritted funnel and washed with ice cold pentane (45 mL). The powder was then placed under high vacuum for 16 hours. ¹H NMR (DMSO D₆, 600 MHz) [symbol] 7.56 (d, J=8.5 Hz, 2H), 7.33 (d, J=8.5 Hz, 2H), 7.20-7.26 (m, 1H), 7.00-7.12 (m, 2H), 3.42-3.52 (m, 1H), 2.66-2.80 (m, 4H).

### Example 21

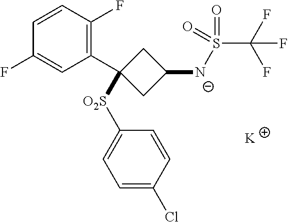

### Potassium[cis-3-[(4-chlorophenyl)sulfonyl]-3-(2,5-difluorophenyl)cyclobutyl][(trifluoromethyl)sulfonyl]azanide

N-[cis-3-[(4-chlorophenyl)sulfonyl]-3-(2,5-difluorophenyl)cyclobutyl]-1,1,1-trifluoromethanesulfonamide (1.2 g, 2.55 mmol) was stirred in anhydrous THF (25.5 mL) at 0° C. and then treated with potassium tert-butoxide (0.29 g, 2.55 mmol). The reaction mixture was stirred at 0° C. for 15 minutes then warmed to ambient temperature and stirred for another 45 minutes. After the reaction was concentrated in vacuo the resultant white powder was recrystallized from a minimal amount of 3:1 IPA:Toluene (400 mL) stirring at 100° C. Once in solution the mixture was filtered through paper and allowed to sit undisturbed at 4° C. for 20 hours. Crystals were harvested by filtration through a glass frit, and washed with cold pentane three times. Residual solvent was removed under vacuum. ¹H NMR (DMSO D₆, 600 MHz) [symbol] 7.56 (d, J=8.5 Hz, 2H), 7.33 (d, J=8.5 Hz, 2H), 7.20-7.26 (m, 1H), 7.00-7.12 (m, 2H), 3.42-3.52 (m, 1H), 2.66-2.80 (m, 4H).

### Example 22

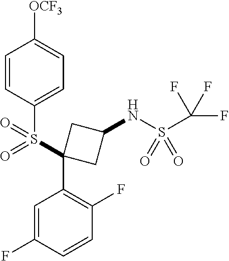

### N-[cis-3-[(4-trifluoromethoxyphenyl)sulfonyl]-3-(2,5-difluorophenyl)cyclobutyl]-1,1,1-trifluoromethanesulfonamide

Prepared as for Example 2, using cis-3-[(4-trifluoromethoxyphenyl)sulfonyl]-3-(2,5-difluorophenyl)cyclobutanamine.

¹H NMR (600 MHZ, CDCl₃) δ 7.56 (m, 2H), 7.27 (m, 2H) 7.088 (m,1H), 6.78 (m, 2H), 6.68 (d, J=10.6 Hz, 1H, NH), 4.30 (m, 1H), 3.31 (m, 2H), 3.18 (m, 2H). MS calculated 603.46 (MNa⁺+CH₃CN), exp 603.0 (MNa⁺+CH₃CN).

### Example 23

### Compound VIB

### 1,1,1-trifluoro-N-(4-[5-fluoro-2-(trifluoromethyl)phenyl]-4-{[4-(trifluoromethyl)phenyl]sulfonyl}cyclohexyl)methanesulfonamide

### Step 1:

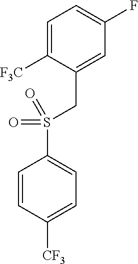

### 4-fluoro-1-(trifluoromethyl)-2-({[4-(trifluoromethyl)phenyl]sulfonyl}methyl)benzene

Sodium sulfite (102 g, 809 mmol) was added to sodium phosphate, dibasic (58 g, 409 mmol) and water (550 ml). 4-(trifluoromethyl)benzenesulfonyl chloride (100 g, 409 mmol) and dioxane (65 ml) were then added to the reaction mixture. The reaction was heated to reflux. After 75 min, the reaction was cooled slightly and a solution of 2-trifluoromethyl-5-fluoro-benzyl bromide (75 g, 292 mmol) in ethanol (95 ml) were added and the mixture was refluxed for 1 hr. The reaction was diluted with water (400 ml) and cooled to 0° C. for 1 hr. The reaction mixture was filtered and the solid was washed with water (2×400 ml) and then with heptane (200 ml). The residue was dried under vacuum to a constant weight and carried to the next step without further purification.

¹H NMR (600 MHz, CDCl₃) δ 7.82 (d, J=8.3 Hz, 2H), 7.74 (d, J=8.4 Hz, 2H), 7.61 (dd, J=8.8, 5.4 Hz, 1H), 7.57 (dd, J=9.1, 2.5 Hz, 1H), 7.21-7.11 (m, 1H), 4.53 (s, 2H).

### Step 2:

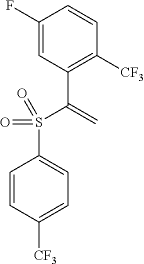

### 1-[5-fluoro-2-(trifluoromethyl)phenyl]ethenyl 4-(trifluoromethyl)phenylsulphone

4-fluoro-1-(trifluoromethyl)-2-({[4-(trifluoromethyl)phenyl]sulfonyl}methyl)benzene (201.5 g, 522 mmol) was dissolved in DMF (1100 ml). Acetic anhydride (75 ml, 795 mmol) was added over about 3 min. The reaction was heated to 60° C. and stirred at 60° C. for 1.5 hr. A second portion of acetic anhydride (105 ml, 1113 mmol) was added dropwise over 1 hr and the reaction was heated at 60° C. for another 5.4 hr. Another portion of acetic anhydride (30 ml) was added and heated for another 5 hr at 60° C. The next day another 10 ml of acetic anhydride was added and heated to 70° C. for 2 hr. The reaction was then cooled to room temperature, water was added dropwise over 30 min. The suspension was cooled to 20° C. and filtered. The residue was washed with water (1×500 ml, and 2×250 ml). The compound was initially dried under vacuum at room temperature and then at 50° C. to a constant weight to provide 1-[5-fluoro-2-(trifluoromethyl)phenyl]ethenyl 4-(trifluoromethyl)phenylsulphone.

¹H NMR (500 MHz, CDCl₃) δ 7.78 (d, J=8.1 Hz, 2H), 7.70 (d, J=8.1 Hz, 2H), 7.65-7.59 (m, 1H), 7.40 (d, J=8.9 Hz, 1H), 7.22 (t, J=8.0 Hz, 1H), 6.87 (s, 1H), 6.08 (s, 1H). MS calculated 421.3 [M+Na]+, exp 421.1 [M+Na]+.

### Step 3:

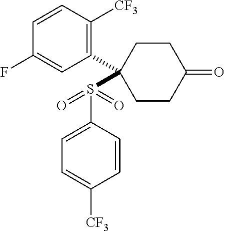

### 4-[5-fluoro-2-(trifluoromethyl)phenyl]-4-{[4-(trifluoromethyl)phenyl]sulfonyl}cyclohexanone

1-[5-fluoro-2-(trifluoromethyl)phenyl]ethenyl 4-(trifluoromethyl)phenylsulphone (127.4 g, 320 mmol) and 2-(trimethylsiloxy)-1,3-butadiene (127.4 g, 895 mmol) were combined in a 500 ml round bottom flask flushed with nitrogen and fitted with a condenser and then placed in a 130° C. preheated oil bath. The reaction was stirred at 130-132° C. for 5 days and then transferred with toluene to a larger round bottom to concentrate. Ethyl acetate (1 L) and 1 N HCl (200 ml) were added and the reaction was stirred at room temperature for 1 hr. First the insoluble material was filtered and then the layers were separated. The organic layer was washed with brine, dried over MgSO4, filtered and concentrated. The crude reaction mixture was dissolved in CH₂Cl₂ and purified by column chromatography on silica gel, eluting with ethyl acetate/heptane to provide 4-[5-fluoro-2-(trifluoromethyl)phenyl]-4-{[4-(trifluoromethyl)phenyl]sulfonyl}cyclohexanone.

¹H NMR (500 MHz, CDCl₃) δ 7.92 (dd, J=9.0 Hz, 6.0, 1H), 7.73 (d, J=8.3 Hz, 2H), 7.63 (d, J=8.2 Hz, 2H), 7.47 (dd, J=11.2, 2.4 Hz, 1H), 7.32-7.27 (m, 1H), 3.02 (dd, J=16.1, 3.5 Hz, 2H), 2.67 (td, J=14.2, 3.8 Hz, 2H), 2.56 (d, J=16.5 Hz, 2H), 2.32-2.21 (m, 2H). MS calculated 491.4 [M+Na]+, exp 491.0 [M+Na]+.

### Step 4:

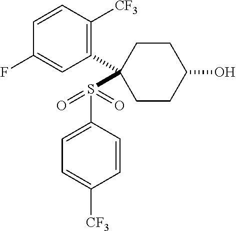

### 4-[5-fluoro-2-(trifluoromethyl)phenyl]-4-{[4-(trifluoromethyl)phenyl]sulfonyl}cyclohexanol

A 1 L 3-neck round bottom flask equipped with a nitrogen bubbler, thermocouple, stir bar and condenser was charged with 4-[5-fluoro-2-(trifluoromethyl)phenyl]-4-{[4-(trifluoromethyl)phenyl]sulfonyl}cyclohexanone (45 g, 96 mmol) in ethanol (464 ml). Next sodium borohydride (9.67 g, 256 mmol) was added and the reaction was stirred for 30 min. The reaction was quenched by adding HCl (200 ml, 2N) slowly over 10 minutes and then the reaction was poured into HCl (2 L, 2N) and extracted with ethyl acetate (3×1 L). The organic layers were washed with brine, dried over MgSO₄, filtered and concentrated to provide 4-[5-fluoro-2-(trifluoromethyl)phenyl]-4-{[4-(trifluoromethyl)phenyl]sulfonyl}cyclohexanol.

¹H NMR (600 MHz, DMSO) δ 8.01 (dd, J=9.0, 6.2 Hz, 1H), 7.92 (d, J=8.3 Hz, 2H), 7.64 (d, J=8.1 Hz, 2H), 7.54-7.46 (m, 1H), 7.35 (dd, J=11.9, 2.4 Hz, 1H), 4.54 (d, J=5.1 Hz, 1H), 3.43 (dt, J=15.6, 5.3 Hz, 1H), 2.71 (d, J=48.9 Hz, 2H), 1.97 (t, J=13.3 Hz, 2H), 1.80 (d, J=20.4 Hz, 2H), 0.91 (d, J=52.4 Hz, 2H). MS calculated 491.4 [M+Na]+, exp 491.0 [M+Na]+

### Step 5:

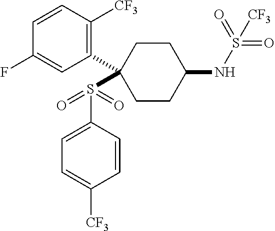

### 1,1,1-trifluoro-N-(4-[5-fluoro-2-(trifluoromethyl)phenyl]-4-{[4-(trifluoromethyl)phenyl]sulfonyl}cyclohexyl)methanesulfonamide

A 2 L 3-neck round bottom flask equipped with a nitrogen bubbler, thermocouple, stir bar, heating mantle and condenser was charged with 4-[5-fluoro-2-(trifluoromethyl)phenyl]-4-{[4-(trifluoromethyl)phenyl]sulfonyl}cyclohexanol (94.7 g, 201 mmol) in THF (1 L). Trifluoromethanesulfonamide (45.0 g, 302 mmol) and triphenylphosphine (79 g, 302 mmol) were added, followed by DIAD (0.059 L, 302 mmol,) which was added dropwise over 20 min. The reaction was heated to 60° C. and stirred for 18 hrs at 60° C. The reaction was concentrated and dissolved in CH₂Cl₂ and purified by column chromatography on silica gel, eluting with ethyl acetate/heptanes to provide 1,1,1-trifluoro-N-(4-[5-fluoro-2-(trifluoromethyl)phenyl]-4-{[4-(trifluoromethyl)phenyl]sulfonyl}cyclohexyl)methanesulfonamide.

¹H NMR (500 MHz, cdcl3) δ 7.84 (dd, J=9.0, 6.1 Hz, 1H), 7.67 (d, J=8.4 Hz, 2H), 7.51 (d, J=8.1 Hz, 2H), 7.34 (dd, J=11.4 Hz, 2.2, 1H), 7.25-7.21 (m, 1H), 5.84 (d, J=7.7 Hz, 1H), 3.79-3.73 (m, 1H), 2.77-2.53 (m, 4H), 2.19-2.05 (m, 2H), 1.79-1.65 (m, 2H). MS calculated 623.5 [M+Na]+, exp 623.7 [M+Na]+

### Biological Activity

Assays to determine the activity of putative GSIs towards processing of APP and Notch include the following:

APP Processing (Assay Quantitates Secreted AβAnalytes from Cell Lines):

The effect of compounds on the abundance of Aβ40 and Aβ42 peptides generated from SH-SY5Y cells expressing amyloid β protein (SP4CT cells) was determined by an AlphaLisa™ assay. Analogous to an ELISA assay, generation of signal in this AlphaLisa™ assay requires “donor” and “acceptor” beads to be brought in close proximity by specific antibody recognition of either Aβ40 or Aβ42 peptides. The assay was accomplished by removing media from compound-treated SP4CT cells to two different microplates, followed by the addition of donor beads conjugated with streptavidin binding a biotinylated anti-amyloid β monocolonal antibody (clone 4G8). Acceptor beads directly conjugated with anti-Aβ40 monoclonal antibody (G210) were added to one microplate and anti-Aβ42 monoclonal antibody (12F4) acceptor beads were added to the other. Abundance of Aβ40 and Aβ42 was directly proportional to the luminescent signal generated following excitation of donor beads by laser light.

### Notch Processing: (Assay Quantitates Notch Intracellular Domain Release in Cell Lines):

A “split-luciferase” assay is used to measure inhibition of gamma secretase-dependent cleavage of the Notch protein. In this assay, HeLa cells were made to express a Notch protein lacking its extracellular domain (NotchΔE) fused to an N-terminal fragment of luciferase. The same cells also expressed a C-terminal fragment of luciferase fused to the immunoglobulin J kappa recombination signal sequence binding protein (RBP). Upon NotchΔE cleavage by gamma secretase, a Notch intracellular domain (NICD)-N terminal luciferase protein is generated which translocates to the nucleus and binds the RBP-C terminal luciferase fusion, bringing two independently nonfunctional halves of luciferase together to form a functional luciferase enzyme. The activity of luciferase in these cells is directly proportional to the amount of gamma secretase-cleaved Notch. Luciferase activity is determined by the standard techniques of luciferin addition to lysed cells and measurement of total luminescence.

### ICD Transactivation (Assay Quantitates Intracellular Domain Release of a Panel of γ-Secretase Substrates in Cell Lines)

A Firefly luciferase based transactivation assay is used to measure inhibition of ε/S3-site cleavage of γ-secretase substrates. This assay involves the use of chimeric substrates harboring a GAL4/VP16 (GVP) transactivation domain fused to the intracellular domain (ICD): APP-GVP, NotchΔE-GVP, E-cadherin-GVP and CD44-GVP. Upon cleavage and release of ICDs, the GVP domain drives the expression of the luciferase gene under the control of the UAS promoter. In this assay, HEK cells were transiently co-transfected with the chimeric substrate along with a UAS promoter driven luciferase and β-galactosidase (transfection control). Upon cleavage by γ-secretase, the released ICD-GVP translocates to the nucleus to drive the expression of the UAS-luciferase gene. The activity of luciferase in these cells is directly proportional to the amount of γ-secretase-cleaved ICDs. Luciferase activity is determined by the standard techniques of luciferin addition to lysed cells and measurement of total luminescence. In addition, to account for the differences in transfection efficiencies an absorbance based β-galactosidase enzyme assay is performed to normalize the luminescence read-out.

### Dosage Example A

Compound IIIA (prepared as described in Sparey et al, Bioorg. Med. Chem. Lett. (2005) 15, 4212-4216) was administered orally to groups of mice (n=10) via gavage in 0.5% methylcellulose by a variety of continuous and intermittent regimens over periods of several weeks, and body weights recorded twice-weekly as a measure of tolerance or otherwise of the drug treatment. Less than 20% loss of body weight after 21 days, with no other overt symptoms, was taken as tolerance, and the results are summarised in the following table:

| Dosing Cycle | Daily dose (mg/Kg) | Tolerated |
|---|---|---|
| Daily (continuous) | 50 | Yes |
| | 100 | No |
| | 150 | No |
| | 200 | No |
| Thrice weekly | 100 | Yes |
| (3 days on, 4 days off) | 200 | No |
| Once weekly | 100 | Yes |
| (1 day on, 6 days off) | 200 | Yes |
| | 300 | Yes |

Clearly, larger doses could be tolerated via an intermittent regimen.

Plasma levels of Aβ were monitored 4 hours, 1 day, 3 days, 4 days and 7 days after a single dose of 300 mg/Kg, and found to be (respectively), 0%, 0%, 0%, 67% and 85% of the baseline (control) level, indicating prolonged therapeutic efficacy from the single dose. Plasma levels of the drug had declined to zero within 4 days of the single dose.

### Dosage Example B

Compound IA (synthesis example 2 herein) was administered orally to groups of rhesus monkeys by continuous and intermittent regimens over periods of days or weeks. Physical signs were recorded and the absence of loose stool, and other overt physical signs, was taken as tolerance. The results are summarised in the following table:

| Dosing Cycle | Daily dose (mg/Kg) | Tolerated |
|---|---|---|
| Daily (continuous, | 3 | No |
| e.g. 3 or more days) | 10 | No |
| | 100 | No |
| Once weekly | 10 | Yes |
| (1 day on, 6 days off) | 50 | Yes |
| | 100 | Yes |
| | 200 | Yes |

While doses of Compound IA equal to or greater than 3 mg/Kg were not tolerated with daily dosing, doses of Compound IA are tolerated via the intermittent regimen and, importantly, CSF levels of Aβ monitored post-dose indicate that doses below 10 mg/Kg do not effect Aβ levels appreciably. Thus, only weekly doses of Compound IA are both tolerated and lower CSF Aβ significantly in rhesus monkey, and by extension in human.

### Dosage Example C

Compound VIA (U.S. Pat. No. 6,890,956) was administered orally to Tg2576 mice at a daily dose of 2.5 mpk or as a weekly dose of 100 mpk. Plasma levels of Aβ40 and Aβ42 were monitored acutely. All results are relative to vehicle-treated controls. The 2.5 mpk QD and 100 mpk produce similar average Aβ inhibition shown in the table below:

Acute studies:

| | |
|---|---|
| 2.5 mpk QD | 100 mpk QW |

| | Aβ40 | Aβ42 | Aβ40 | Aβ42 |
|---|---|---|---|---|
| Weekly av. inhibition | 58% | 47% | 51% | 44% |
| Max. inhibition | 68% | 55% | 81% | 67% |

These two doses were then chosen for a 6 month long term studies to evaluate plaque reduction. The levels of insoluble Aβ40 and Aβ42 in the brain were determined, giving the following results:

Long term studies (insoluble Aβ):

| | 2.5 mpk QD | 100 mpk QW |
|---|---|---|
| Aβ40 | −46% | −41% |
| Aβ42 | −47% | −56% |

All results are relative to vehicle-treated controls. Weekly dosing was found to be at least as effective as daily dosing, but with reduced risk of GI toxicity.

### Dosage Example D

Compound VIB (Example 23 herein) was administered orally to rhesus monkeys once-weekly at a dose of 30 mpk and levels of Aβ40 and Aβ42 in the cerebrospinal fluid (CSF) were monitored. Over a 3 week period, average reductions of approximately 40% in CSF Aβ40 and Aβ42 levels were observed, relative to vehicle-treated controls, with no evidence of GI toxicity. (However, the study was terminated after 3 weeks as a result of respiratory side-effects unconnected with Notch signalling.)

1. A method for treating or preventing a disease involving deposition of β-amyloid (Aβ) in the brain which comprises administering to a patient in need thereof a therapeutically effective amount of a gamma-secretase inhibitor (GSI) by an intermittent dosing regimen.

2. A method according to claim 1 wherein the disease is Alzheimer's disease.

3. A method according to claim 1 wherein the intermittent dosing regimen comprises a repeating cycle of GSI administration on 1 to 3 consecutive days followed by at least 4 days of rest.

4. A method according to claim 3 wherein the GSI is administered on 3 consecutive days followed by 4 days of rest.

5. A method according to claim 3 wherein the GSI is administered on 1 day followed by 6 days of rest.

6. A method according to claim 1 wherein the GSI is a compound of formula I: 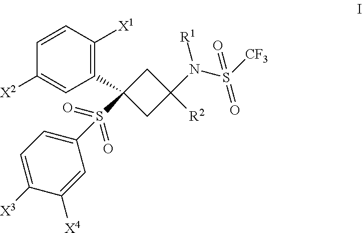
or pharmaceutically acceptable salt thereof, wherein:
X¹ is selected from the group consisting of: F and CN;
X² is selected from the group consisting of: F, Cl and CN;
X³ is selected from the group consisting of: F, Br, Cl, CN, CF₃, OCF₃, C(O)—OCH₃ and S—CH₃;
X⁴ is selected from the group consisting of: H, F and Cl;
R¹ is selected from the group consisting of:
(a) H,
(b) CH₃,
(c) —(CH₂)ₙ—OR³;
(d) —(CH₂)ₙ—C(O)—OR⁴ and
(e) —SO₂—CF₃;
R² is H or CH₃ when the compound of formula I is in the cis configuration, otherwise R² is H;
R³ is a five- or six-membered non-aromatic heterocycle having one oxygen heteroatom;
R⁴ is H or CH₃; and
n is 1 to 4.

7. A method according to claim 6 wherein X¹ and X² are F; X³ is Cl; and X⁴ is H.

8. A method according to claim 6 wherein the GSI is a compound of formula: 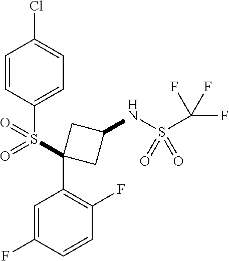
or a pharmaceutically acceptable salt thereof.

9. A method according to claim 1 wherein the GSI is a compound of formula II: 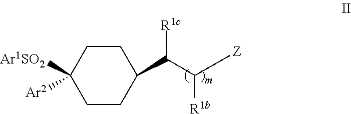
wherein:
m is 0 or 1;
Z represents CN, OR^{2a}, CO₂R^{2a} or CON(R^{2a})₂;
R^{1b} represents H, C₁₋₄alkyl or OH;
R^{1c} represents H or C₁₋₄alkyl;
Ar¹ represents phenyl or pyridyl, either of which bears 0-3 substituents independently selected from halogen, CN, NO₂, CF₃, OH, OCF₃, C₁₋₄alkoxy or C₁₋₄alkyl which optionally bears a substituent selected from halogen, CN, NO₂, CF₃, OH and C₁₋₄alkoxy;
Ar² represents phenyl which is substituted in the 2- and 5-positions with halogen;
R^{2a} represents H, C₁₋₆alkyl, C₃₋₆cycloalkyl, C₃₋₆cycloalkylC₁₋₆alkyl, C₂₋₆alkenyl, any of which optionally bears a substituent selected from halogen, CN, NO₂, CF₃, OR^{2b}, CO₂R^{2b}, N(R^{2b})₂, CON(R^{2b})₂, Ar and COAr; or R^{2a} represents Ar; or two R^{2a} groups together with a nitrogen atom to which they are mutually attached may complete an N-heterocyclyl group bearing 0-4 substituents independently selected from ═O, ═S, halogen, C₁₋₄alkyl, CN, NO₂, CF₃, OH, C₁₋₄alkoxy, C₁₋₄alkoxycarbonyl, CO₂H, amino, C₁₋₄alkylamino, di(C₁₋₄alkyl)amino, carbamoyl, Ar and COAr;
R^{2b} represents H, Cₗ₋₆alkyl, C₃₋₆cycloalkyl, C₃₋₆cycloalkylC₁₋₆alkyl, C₂₋₆alkenyl, any of which optionally bears a substituent selected from halogen, CN, NO₂, CF₃, OH, C₁₋₄alkoxy, C₁₋₄alkoxycarbonyl, CO₂H, amino, C₁₋₄alkylamino, di(C₁₋₄alkyl)amino, carbamoyl, Ar and COAr; or R^{2b} represents Ar; or two R^{2b} groups together with a nitrogen atom to which they are mutually attached may complete an N-heterocyclyl group bearing 0-4 substituents independently selected from ═O, ═S, halogen, C₁₋₄alkyl, CN, NO₂, CF₃, OH, C₁₋₄alkoxy, C₁₋₄alkoxycarbonyl, CO₂H, amino, C₁₋₄alkylamino, di(C₁₋₄alkyl)amino, carbamoyl, Ar and COAr;
Ar represents phenyl or heteroaryl bearing 0-3 substituents selected from halogen, C₁₋₄alkyl, CN, NO₂, CF₃, OH, C₁₋₄alkoxy, C₁₋₄alkoxycarbonyl, amino, C₁₋₄alkylamino, di(C₁₋₄alkyl)amino, carbamoyl, C₁₋₄alkylcarbamoyl and di(C₁₋₄alkyl)carbamoyl;
or a pharmaceutically acceptable salt thereof.

10. A method according to claim 9 wherein the GSI is cis-4-[(4-chlorophenyl)sulfonyl]-4-(2,5-difluorophenyl)cyclohexanepropanoic acid or the sodium salt thereof.

11. A method according to claim 1 wherein the GSI is a compound of formula (III): 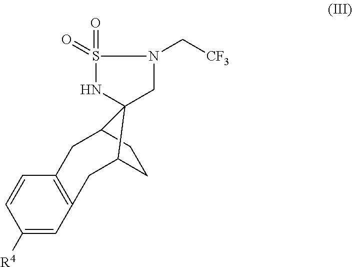
or a pharmaceutically acceptable salt thereof;
wherein R⁴ is —CH═CHCH₂N(R¹⁶)₂ where —N(R¹⁶)₂ is morpholin-4-yl, 4-trifluoromethylpiperidin-1-yl, 5-aza-2-oxabicyclo[2.2.1]hept-1-yl, 4,4-difluoropiperidin-1-yl, 4-hydroxy-4-trifluoromethylpiperidin-1-yl, 4-methylpiperidin-1-yl, 3-oxo-4-phenylpiperazin-1-yl, 3-oxo-4-cyclohexylpiperazin-1-yl, 3-oxo-piperazin-1-yl, N-(tetrahydrofuran-3-yl)amino, N-methyl-N-(tetrahydrofuran-3-yl)amino, N-(tetrahydropyran-4-yl)amino, N-methyl-N-(tetrahydropyran-4-yl)amino, N-(dioxanylmethyl)amino, N-[(tetrahydropyran-2-yl)methyl]amino, 3-hydroxypiperidin-1-yl, 5-aza-2-oxabicyclo[5.4.0]undeca-7,9,11-trien-5-yl, 2-(phenoxymethyl)morpholin-4-yl, N-[(4-phenylmorpholin-2-yl)methyl]amino, 3,3-difluoropyrrolidin-1-yl, N-(2,2,2-trifluoroethyl)amino, or 3-(pyridin-3-yl)pyrrolidin-1-yl.

12. A method according to claim 1 wherein the GSI is a compound of formula IV: 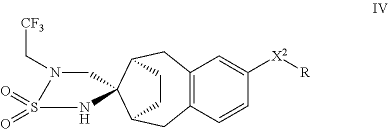
or a pharmaceutically acceptable salt thereof;
wherein X² is a bivalent pyrazole, imidazole, triazole, oxazole, isoxazole, thiazole, isothiazole, thiadiazole or 1,3,4-oxadiazole residue optionally bearing a hydrocarbon substituent comprising 1-5 carbon atoms which is optionally substituted with up to 3 halogen atoms; and R is selected from:
(i) CF₃ or an alkyl group of up to 6 carbon atoms, optionally substituted with halogen, CF₃, CHF₂, CN, OH, CO₂H, C₂₋₆acyl, C₁₋₄alkoxy or C₁₋₄alkoxycarbonyl;
(ii) a non-aromatic heterocyclic group comprising up to 7 ring atoms of which up to 3 are chosen from N, O and S and the remainder are carbon, bearing 0-3 substituents independently selected from oxo, halogen, CN, C₁₋₆alkyl, OH, CF₃, CHF₂, CH₂F, C₂₋₆acyl, CO₂H, C₁₋₄alkoxy and C₁₋₄alkoxycarbonyl;
(iii) phenyl or 6-membered heteroaryl, either of which bears 0-3 substituents independently selected from halogen, CF₃, CHF₂, CH₂F, NO₂, CN, OCF₃, C₁₋₆alkyl and C₁₋₆alkoxy; and
(iv) N(R^{a})₂ where each R^{a} independently represents H or C₁₋₆alkyl which is optionally substituted with halogen, CF₃, CHF₂, CN, OH, C₁₋₄alkoxy or C₁₋₄alkoxycarbonyl.

13. A method according to claim 12 wherein X² represents 5-substituted-thiazol-2-yl, 5-substituted-4-methylthiazol-2-yl, 5-substituted-1-methylpyrazol-3-yl, 1-substituted-imidazol-4-yl or 1-substituted-1,2,4-triazol-3-yl; and R represents 4-fluorophenyl, 4-chlorophenyl or 3,4-difluorophenyl.

14. A method according to claim 1 wherein the GSI is a compound of formula: 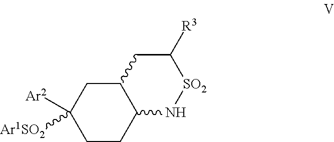
wherein the bonds indicated by wavy lines are mutually cis with respect to the cyclohexane ring;
R³ represents H or a hydrocarbon group of up to 10 carbon atoms, optionally substituted with CF₃, CHF₂, halogen, CN, OR⁵, COR⁵, CO₂R⁵, OCOR⁶, N(R⁵)₂, CON(R⁵)₂ or NR⁵COR⁶;
R⁵ represents H or C₁₋₄alkyl;
R⁶ represents C₁₋₄alkyl; and
Ar¹ and Ar² independently represent phenyl or heteroaryl, either of which bears 0-3 substituents independently selected from halogen, CN, NO₂, CF₃, CHF₂, OH, OCF₃, CHO, CH═NOH, C₁₋₄alkoxy, C₁₋₄alkoxycarbonyl, C₂₋₆acyl, C₂₋₆alkenyl and C₁₋₄alkyl which optionally bears a substituent selected from halogen, CN, NO₂, CF₃, OH and C₁₋₄alkoxy;
or a pharmaceutically acceptable salt thereof.

15. A method according to claim 14 wherein the GSI is selected from:
(4aRS,6RS,8aSR)-6-(2,5-difluorophenyl)-6-{[4-(trifluoromethyl)phenyl]sulfonyl}octahydro-1H-2,1-benzothiazine 2,2-dioxide;
(3R,4aS,6S,8aR)-6-(2,5-difluorophenyl)-3-ethyl-6-{[4-(trifluoromethyl)phenyl]sulfonyl}octahydro-1H-2,1-benzothiazine 2,2-dioxide;
(3S,4aS,6S,8aR)-6-(2,5-difluorophenyl)-3-ethyl-6-{[4-(trifluoromethyl)phenyl]sulfonyl}octahydro-1H-2,1-benzothiazine 2,2-dioxide;
(3RS,4aRS,6RS,8aSR)-6-(2,5-difluorophenyl)-3-isopropyl-6-{[4-(trifluoromethyl)phenyl]sulfonyl}octahydro-1H-2,1-benzothiazine 2,2-dioxide;
(3SR,4aRS,6RS,8aSR)-6-(2,5-difluorophenyl)-3-isopropyl-6-{[4-(trifluoromethyl)phenyl]sulfonyl}octahydro-1H-2,1-benzothiazine 2,2-dioxide; and
(3R,4aS,6S,8aR)-6-[(4-chlorophenyl)sulfonyl]-6-(2,5-difluorophenyl)-3-ethyloctahydro-1H-2,1-benzothiazine 2,2-dioxide;
and the pharmaceutically acceptable salts thereof.

16. A method according to claim 1 wherein the GSI is a compound of formula: 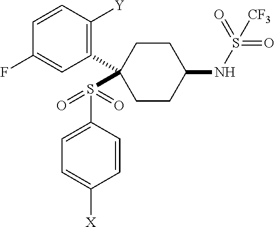
or a pharmaceutically-acceptable salt thereof,
wherein X is Cl and Y is F; or X and Y are both CF₃.
